(19) United States
(12) Patent Application Publication
(10) Pub. No.: US 2004/0238656 A1
(43) Pub. Date: Dec. 2, 2004

(54) Fragrant pouch

(76) Inventor: Chien-Chung Wang, Taipei (TW)

Correspondence Address: Chien-Chung Wang, PO Box 82-144, Taipei, TW

(21) Appl. No.: 10/449,004

(22) Filed: Jun. 2, 2003

(51) Int. Cl. A61L 9/04; A24F 25/00

(52) U.S. Cl. 239/34

(57) ABSTRACT

A fragrant pouch which includes an inner bag made of one-way permeable film containing liquid fragrant essence oil or perfume; an outer bag made of non-woven fabric with both bags laminated together by fusion or by high frequency into various types of form as desired; an opening to the pouch, and a hook to the opening; that can be placed flat in drawer and shoes or hung up in car, closet and closet for the fragrant essence oil or perfume to seep out of the inner bag through the non-woven outer bag for slow volatilization and emission into the ambient space.

### BACKGROUND OF THE INVENTION

(a) Field of the Invention

The present invention is related to a structure of fragrant pouch, and more particularly, to one that can be placed or hung up at any location as desired to achieve emission of fragrance and the purpose of compliance with environment protection.

(b) Description of the Prior Art

There are many types of fragrant emitter generally available in the market. One type among others as illustrated in FIG. 1 of the accompanying drawings of the present invention is comparatively popular. Wherein, solid state or multiple particles of fragrant essence materials are contained in a canister 1, and one or multiple openings is provided on the surface of the canister 1 for the fragrant essence to external emit through the opening 11. Air permeable fabric or non-woven fabric is provided to the opening 11 of the canister to slow down the emission rate. Another type of fragrant emitter as illustrated in FIG. 2 is related to a bottle 2 (usually a glass bottle) containing fragrant essence oil or perfume 3 to externally volatilize and emit the fragrance. However, disregarding the filling method, the fragrant emitters of the prior art are found with the following flaws:

1. Either the canister 1 or the bottle 2 when used up with its contents is disposed to become refuses that are difficult to process thus resulting in environmental problem.

2. Both of the canister 1 and the bottle 2 are made in comparatively larger sizes that limit their places of application, and the bottle 2 is also vulnerable to breaking into pieces to present safety problem.

Furthermore, once the canister 1 or the bottle 2 is opened up, it immediately contacts the ambient air to accelerate its volatilization to shorten its service life.

### SUMMARY OF THE INVENTION

The primary purpose of the present invention is to provide a fragrant pouch essentially comprised of an inner bag made of one-way permeable film containing liquid fragrant oil or perfume, and an outer bag made of non-woven fabric with both bags to be laminated by high frequency process to form a fragrant pouch. An opening is provided at where appropriately on the pouch and a hook is provided to the opening so that the pouch can be hung up on in the bathroom, car, closet, or simply place flat in a drawer or shoes for the fragrant essence oil or perfume to seep through the one-way permeable film of the inner bag then through the non-woven outer bag to slowly emit into the ambient air for achieving a fragrant surrounding and environmental compliance.

Another purpose of the present invention is to provide a fragrant pouch that can be made into various forms and printed on its surface patterns for decoration or advertisement results in the process of fusing or sealing by high frequency the inner bag to the outer bag.

Another purpose yet of the present invention is to provide a fragrant pouch that the fragrant essence oil or perfume is released only from inside out due to the one-way permeable film so that the seepage by outer liquid is prevented for warranting longer service life of the fragrant pouch.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of a fragrant emitter of the prior art.

FIG. 2 is a perspective view of another perspective view of another fragrant emitter of the prior art.

FIG. 3 is a perspective view of a preferred embodiment of the present invention.

FIG. 4 is a sectional view of the preferred embodiment of the present invention.

FIG. 5 is a schematic view showing an application of the preferred embodiment of the present invention.

FIG. 6 is a schematic view showing another application of the preferred embodiment of the present invention.

FIG. 7 is a schematic view showing another preferred embodiment of the present invention.

FIG. 8 is a schematic view showing another preferred embodiment yet of the present invention.

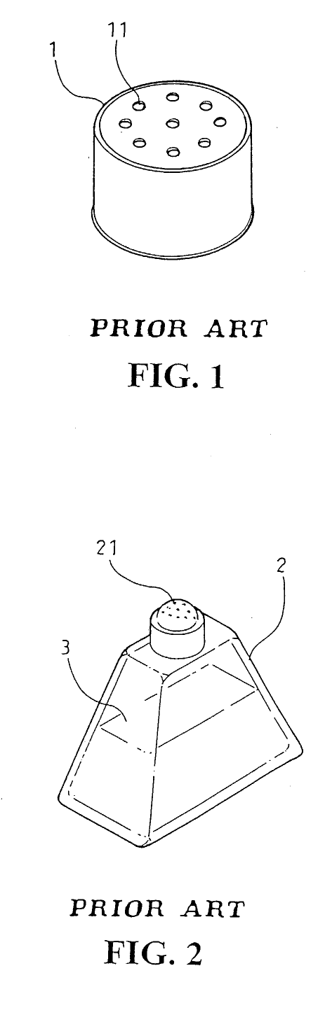

### DETAILED DESCRIPTION OF THE PREFERRED EMBODIMENT

The following descriptions are of exemplary embodiments only, and are not intended to limit the scope, applicability or configuration of the invention in any way Rather, the following description provides a convenient illustration for implementing exemplary embodiments of the invention. Various changes to the described embodiments may be made in the function and arrangement of the elements described without departing from the scope of the invention as set forth in the appended claims.

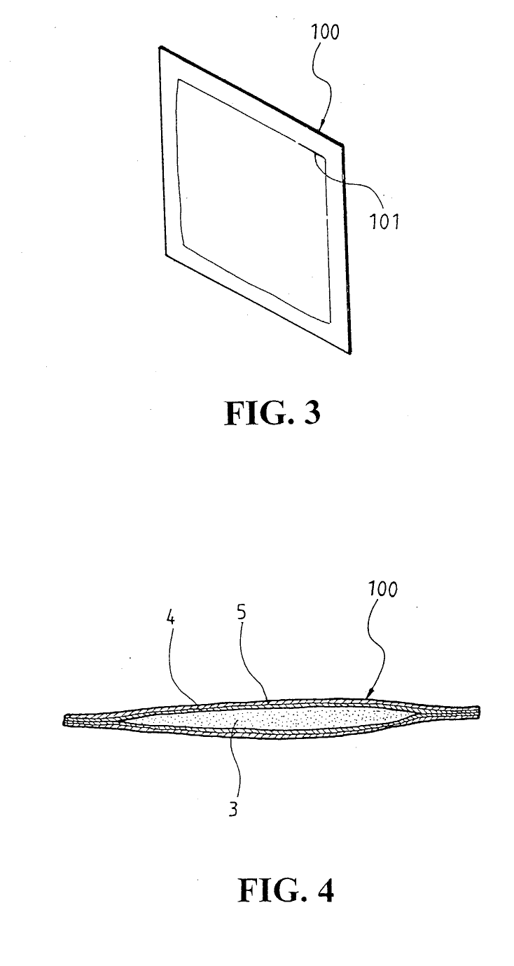

Referring to FIGS. 3 and 4, a preferred embodiment of the present invention is essentially comprised of an inner bag 4 made of one-way permeable film (PU preferred) containing liquid fragrant essence oil or perfume 3; an outer bag 5 made of non-woven fabric laminated with the inner bag 4 by fusion or by high frequency into a fragrant pouch 100 in a form as desired; an opening 101 provided at where appropriately on the fragrant pouch 100. The fragrant pouch 100 is placed flat on the desk, in a drawer and in a pair of shoes, or when provided with a hook to the opening 101 hung up in a car, closet and bathroom for the fragrant essence oil or the perfume 3 to seep out of the inner bag 4 of the one-way permeable film and the non-woven outer bag 5 to slowly volatilize into the ambient space.

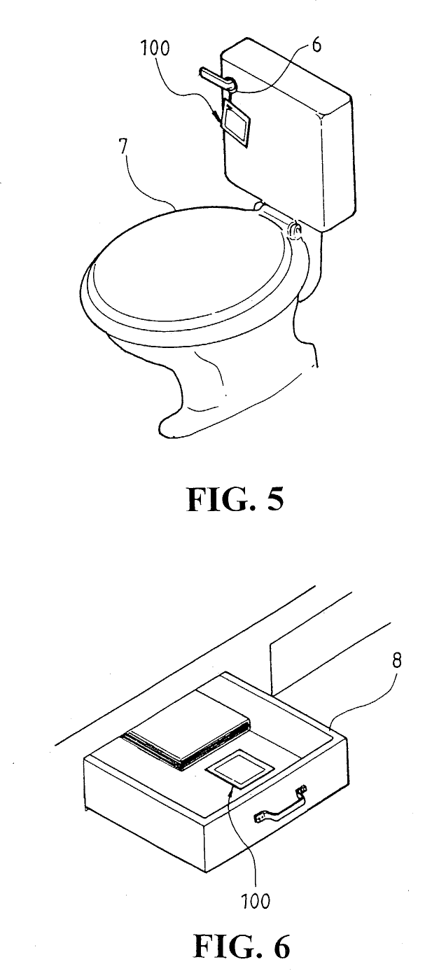

Now referring to FIG. 5, a hook 6 is attached to the opening 101 of the fragrant pouch for the preferred embodiment of the present invention to be hung up to a handle 7 of a cistern of a toilet bowl in the bathroom to rid off the odor. As illustrated in FIG. 6, the fragrant pouch 100 is placed flat in a drawer for one to enjoy the fragrance whenever he or she opens up the drawer.

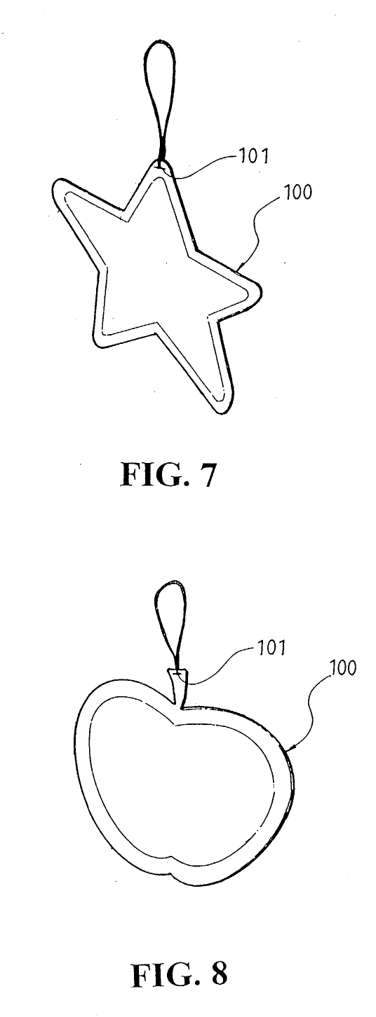

While laminating the inner bag 4 and the outer bag 5, the resultant pouch 100 may be made into various forms in an asterisk as illustrated in FIG. 7 or in a fruit shape as illustrated in FIG. 8 for the fragrant pouch to become even more attractive while on one or both sides of the fragrant pouch 10, patterns and/or letters are printed for decoration or advertisement purpose.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A fragrant pouch comprising an inner bag made of one-way permeable film containing liquid flagrant essence oil or perfume and an outer bag made of non-woven fabric with both bags laminated together by fusion or by high frequency into various types of form as desired that can be placed flat in drawer and shoes or hung up in car, closet and closet for the fragrant essence oil or perfume to seep out of the inner bag through the non-woven outer bag for slow volatilization and emission into the ambient space.

2. A fragrant pouch as claimed in claim 1, wherein, an opening is provided at where appropriately on the laminated inner and outer bags, and a hook is provided to the opening for the fragrant pouch to be hung up in various places as desired.
